(19) United States
(12) Patent Application Publication
(10) Pub. No.: US 2005/0272131 A1
(43) Pub. Date: Dec. 8, 2005

(54) Methods to make and use antibodies of improved cross-reactivity

(76) Inventors: Barbara A. Bartol, Gorham, ME (US); Roger N. Piasio, Cumberland, ME (US)

Correspondence Address: T. D. FOSTER, 12760 HIGH BLUFF DRIVE, SUITE 300, SAN DIEGO, CA, 92130, US

(21) Appl. No.: 11/069,079

(22) Filed: Mar. 1, 2005

Related U.S. Application Data
(60) Provisional application No. 60/549,394, filed on Mar. 2, 2004.

(51) Int. Cl. C12P 21/04

(52) U.S. Cl. 435/70.21; 530/388.4

(57) ABSTRACT

The present invention relates to methods of generating antibodies of improved cross-reactivity against antigens that give rise to immunotypic variations in infectious organisms. The methods include immunizing animals with multiple immunogen preparations that are derived from the antigen of interest. The present invention also includes methods of use of the antibodies of improved cross-reactivity, and assays and kits for employing such methods.

### BACKGROUND OF THE INVENTION

Field of the Invention

The present invention relates generally to the fields of immunology and molecular biology, and particularly to methods for generating and using antibodies that are cross-reactive against antigens that give rise to immunotypic variability in infectious organisms.

### BRIEF SUMMARY OF THE INVENTION

The present invention relates to methods to make and use antibodies of improved cross-reactivity for infectious organisms that exist in more than one immunotype. In particular, the present invention relates to the use of multiple immunogen preparations, wherein the immunogen is derived from variations of the antigen that gives rise to the immunotypic variations of the infectious organism of interest. The present invention also discloses and claims methods of use for the selected antibodies of improved cross-reactivity.

A first aspect of the present invention includes a method for generating at least one antibody having improved cross-reactivity for an infectious organism that exists in more than one immunotype, wherein the immunotypes are due to at least one antigenic variation.

A second aspect of the present invention includes methods for detecting an infectious organism that exists in more than one immunotype, wherein the immunotypes are due to at least one antigenic variation. These methods can utilize any suitable antibody of improved cross-reactivity as disclosed in the present invention.

A third aspect of the present invention includes kits for detecting an infectious organism that exists in more than one immunotype. Such kits utilize the second method of the present invention.

A fourth aspect of the present invention includes a second method for generating at least one antibody having improved cross-reactivity for an infectious organism that exists in more than one immunotype, wherein the immunotypes are due to at least one antigenic variation.

### BRIEF DESCRIPTION OF THE DRAWINGS

FIG. 1 depicts a representative example of a silver-stained polyacrylamide gel run under denaturing electrophoretic conditions to separate isolated outer membrane protein 2 (OMP2) preparations from different strains of non-typeable Haemophilus influenzae. Lanes 1 and 9 contain molecular weight markers as indicated by the molecular weights to the left of the gel. Lane 2 contains OMP2 from strain 19418. Lanes 3, 5, 7, 8, and 10 contain OMP2 from strain 49401. Lane 4 contains OMP2 from strain 53600. Lane 6 contains OMP2 from strain 51997.

FIG. 2 depicts results of ELISA titer assays using plates coated with intact bacterial cells of eight Haemophilus influenzae strains, including the typeable H. influenzae strains 9006 (type a), 9008 (type d), 10211 (type b), and 51654 (type b). Test bleeds were from 7 Jul. 2003. The response of antiserum from the three rabbits that most strongly responded to each immunization program was compared in a titration assay against whole cells of the eight Haemophilus influenzae strains. Higher overall titers and higher overall cross-reactivity were observed for antibodies generated from the 51997/49401/49766 OMP2 multiple immunogen program than was observed for antisera from the 51997 OMP2 single immunogen program.

FIG. 3 depicts representative ELISA assay results of one concentration (0.5 micrograms per milliliter) of affinity-purified anti-OMP2 antibodies purified from the test bleeds of 5 Jul. 2003. ELISA assays were carried out on Immulon-4 plates coated with purified OMP2 proteins from six NTHi strains (51997, 49766, 49401, 53600, 19418, and 43163) at 20 nanograms per well. Affinity-purified anti-OMP2 antibodies were allowed to bind to the immobilized antigens, and bound anti-OMP2 antibody was detected with goat anti-rabbit IgG conjugated to horseradish peroxidase. Plates were developed with 3,3′,5,5′-tetramethylbenzidine and absorbance measured at 630 nanometers. Greater overall signal was generally observed for antibodies generated from the 51997/49401/49766 OMP2 multiple immunogen program than from the 51997 OPM2 single immunogen program.

FIG. 4 depicts ELISA assay results for the affinity-purified antibodies from the test bleeds of 5 Jul. 2003. Microtiter plates were coated with intact non-typeable Haemophilus influenzae cells (strains 49766, 53600, and 49401). At cell concentrations of about 10⁷ to about 10⁸ cells per well, the observed signals were stronger for antibodies generated from the 51997/49401/49766 OMP2 multiple immunogen program than for antibodies generated from the 51997 OPM2 single immunogen program. At cell concentrations of about 10⁴ to about 10⁶ cells per well, antibody was in excess, resulting in about equivalent observed signals.

### DETAILED DESCRIPTION OF THE INVENTION

Introduction

The present invention recognizes that certain infectious organisms exist in more than one immunotype, wherein the immunotypes are due to variation found in at least one antigen of the infectious organism. Immunotypic variation can result in difficulties in immunochemically detecting the presence or measuring levels of such infectious organisms, since often an antibody or antibodies do not have sufficient cross-reactivity or sensitivity to detect all or substantially all of the immunotypic strains of interest that may be present in a sample. It is often desirable to detect or monitor the presence or levels of such infectious organisms, regardless of immunotypic heterogeneity in the sample. Immunotypic variation can also lead to challenges in development of vaccines for pathogens that exist in multiple immunotypes or are capable of relatively quick mutation from one immunotype to another. For these reasons it is of interest to be able to efficiently produce antibody preparations that possess the desired cross-reactivity and sensitivity to multiple immunotypes. Such antibodies can be of use in immunochemical diagnostics, affinity isolation of antigens, or antigen labelling (for example, in situ labelling), in studies of antigen variation, evolution, or epidemiology, in vaccine development, or in the prophylaxis or treatment of an infectious disease in a subject. One highly desirable use of such antibodies of improved cross-reactivity and sensitivity is in the immunochemical diagnosis of infectious disease states in human subjects.

The present invention provides methods of generating and selecting antibodies of improved cross-reactivity for infectious organisms that exist in more than one immunotype, and methods and kits for detecting such infectious organisms.

As a non-limiting introduction to the breadth of the present invention, the present invention includes several general and useful aspects, including:
1) A method for generating at least one antibody having improved cross-reactivity for an infectious organism that exists in more than one immunotype, wherein the immunotypes are due to at least one antigenic variation, the method including the steps of: (a) providing multiple immunogen preparations derived from the at least one antigenic variation; (b) immunizing at least one animal with the multiple immunogen preparations; and (c) selecting at least one antibody from the immunized at least one animal; wherein the selected at least one antibody is of improved cross-reactivity for the infectious organism, relative to antibodies from animals immunized with a single immunogen derived from the at least one infectious organism.
2) A method to detect an infectious organism that exists in more than one immunotype, wherein the immunotypes are due to at least one antigenic variation, the method including the steps of (a) providing a sample suspected of containing the at least one antigenic variation; (b) providing at least one antibody generated by the first method of the present invention; (c) contacting the sample with the at least one antibody, under conditions that allow the at least one antibody to bind to and form a complex with the at least one antigenic variation; and (d) detecting the complex, wherein the detection is positive if concentration of the infectious organism in the sample is greater than or equal to than a reference concentration, and the detection is negative if concentration of the infectious organism in the sample is less than the reference concentration.
3) A kit for performing the second method of the invention, for detecting an infectious organism that exists in more than one immunotype.
4) A method for generating a selection of antibodies having improved cross-reactivity for an infectious organism that exists in more than one immunotype, wherein the immunotypes are due to at least one antigenic variation, the method including the steps of: (a) providing multiple immunogen preparations derived from the at least one antigenic variation; (b) immunizing multiple groups of animals, wherein each of the groups comprises at least one animal, and wherein each of the animals is immunized with a single immunogen preparation; (c) selecting at least one antibody from each of the groups; and (d) combining the selected antibodies, wherein the combination of selected antibodies is of improved cross-reactivity for the infectious organism. Antibodies generated by the fourth method of the invention may be employed in the methods to detect an infectious organism and in the kits of the invention.

Further objectives and advantages of the present invention will become apparent as the description proceeds and when taken in conjunction with the accompanying drawings. To gain a full appreciation of the scope of the present invention, it will be further recognized that various aspects of the present invention can be combined to make desirable embodiments of the invention.

Throughout this application various publications are referenced. The disclosures of these publications are hereby incorporated by reference, in their entirety, in this application. Citations of these documents are not intended as an admission that any of them are pertinent prior art. All statements as to the date or representation as to the contents of these documents is based on the information available to the applicant and does not constitute any admission as to the correctness of the dates or contents of these documents.

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. The technical terms used herein have their ordinary meaning in the art that they are used, as exemplified by a variety of technical dictionaries. Where a term is provided in the singular, the inventor also contemplates the plural of that term. The nomenclature used herein and the procedures described below are those well known and commonly employed in the art. Where there are discrepancies in terms and definitions used in references that are incorporated by reference, the terms used in this application shall have the definitions given herein. Other technical terms used herein have their ordinary meaning in the art that they are used, as exemplified by a variety of technical dictionaries (for example, Chambers Dictionary of Science and Technology, Peter M. B. Walker (editor), Chambers Harrap Publishers, Ltd., Edinburgh, UK, 1999, 1325 pp.). The inventors do not intend to be limited to a mechanism or mode of action. Reference thereto is provided for illustrative purposes only.

I. A Method for Generating Antibodies of Improved Cross-Reactivity

The present invention includes a method for generating at least one antibody having improved cross-reactivity for an infectious organism that exists in more than one immunotype, wherein the immunotypes are due to at least one antigenic variation, the method including the steps of (a) providing multiple immunogen preparations derived from the at least one antigenic variation; (b) immunizing at least one animal with the multiple immunogen preparations; and (c) selecting at least one antibody from the immunized at least one animal; wherein the selected at least one antibody is of improved cross-reactivity for the infectious organism, relative to antibodies from animals immunized with a single immunogen derived from the at least one infectious organism.

The first method of the present invention may be applied to any infectious organism that exists in more than one immunotype. Suitable infectious organisms include bacteria (including mycoplasmas), viruses, and eukaryotic pathogens such as, but not limited to, pathogenic fungi and protists. More preferably, the first method of the present invention may be applied to pathogens that infect vertebrates, invertebrates, or plants. Most preferably, the method may be applied to pathogens that infect humans, mammals, birds, fish, insects, or plants.

By immunotype is meant a type, subtype, strain, or variant of the infectious organism that can be immunologically defined, that is, that can be distinguished by use of antibodies. Immunotypes encompass, but are not limited to, serological variants or “serovars”, wherein serological differentiation is generally performed on whole organisms. Immunotypic determination may be made on whole or intact organisms, on disrupted or lysed organisms, or on isolated components of organisms (for example, on isolated cellular components or isolated carbohydrate or proteinaceous antigens). Immunotypic determination may be made on antigens as they naturally occur, or on antigens that require chemical, physical, biological, or enzymatic modification to, for example, become available for interaction with an antibody.

Immunotypic variation is due to at least one antigenic variation, that is to say, variation found in at least one antigen of the infectious organism. Thus, the at least one antigenic variation results in at least two distinguishable immunotypes. Antigens can include, but are not limited to, peptides, proteins, carbohydrates, lipids, glycoproteins, glycolipids, lipoproteins, lipopolysaccharides, nucleic acids, and combinations thereof. Antigens can include intact molecules, fragments of molecules, and multi-molecular complexes. Antigenic variation can be due to one or more differences in any characteristic of the at least one antigen, such as, but not limited to, molecular weight or size, specific amino acid sequences, specific combinations of multiple amino acid sequences (which need not be contiguous sequences), glycosylation, lipidation, degree of monomer association (for example, monomer, dimer, trimer, and so forth), phosphorylation, charge, degree of membrane association, availability or exposure to antibody, presence or absence of co-factors, and the like. In some cases, antigenic variation can be due to the absence or presence of the at least one antigen. In some cases, a single antigen that exists in more than one antigenic variation (for example, an individual protein that occurs in more than one antigenic amino acid sequence or an antigen that occurs with different glycosylation patterns) may be used to determine immunotypes of a single species of infectious organism. In some cases, more than one antigen (for example, a combination of individual epitopes, peptides, proteins, carbohydrates, or other antigens) may be used to determine immunotypes of a single species of infectious organism.

The following are non-limiting examples of immunotypes of infectious organisms:
1. Non-typeable Haemophilus influenzae strains can be immunotyped by antigenic differences of the major outer membrane protein, OMP2, a surface antigen (Haase et al. (1994) Infect. Immun., 62:3712-3722; Groeneveld et al. (1989) Infect. Immun., 57:3038-3044).
2. Streptococcus pneumoniae can be immunotyped by antigenic variations of capsular polysaccharide antigens or by antigenic variations of pneumococcal surface protein A (PspA) (Crain et al. (1990) Infect. Immun., 58:3293-3299).
3. Neisseria gonorrhoeae can be immunotyped by antigenic variations of Protein I (Kohl et al. (1990) Eur. J. Epidemiol., 6:91-95).
4. Neisseria meningitidis can be immunotyped by antigenic variations of lipopolysaccharide (LPS) (Jennings et al. (1999) Microbiol., 145:3013-3021).
5. Pseudomonas aeruginosa can be immunotyped by antigenic variations of outer membrane protein F (Hughes et al. (1992) Infect. Immun., 60:3497-3503), or by antigenic variations of O side chains of lipopolysaccharide (Goldberg et al. (1992) Proc. Nat'l. Acad. Sci. USA, 89:10716-10720).
6. A number of serotypes of the influenza A virus are distinguished by differences in their hemagglutinin (Plotkin et al. (2002) Proc. Nat'l. Acad. Sci. USA, 99:6263-6368) and neuramimidase (Colman (1992) Immunol. Cell Biol., 70:209-214) surface glycoprotein antigens.
7. Human immunodeficiency virus type I (HIV-1) can be immunotyped by multiple antigenic variations, consisting of antigenic variations of several peptide epitopes from the V3 region of the 120-kDa envelope glycoprotein (gp120) (Zolla-Pazner et al. (1999) J. Virol., 73:4042-4051). Monoclonal antibodies specific for gp120 V3 epitopes show extensive cross-reactivity across genotypic clades, demonstrating that HIV-1 gp120 V3 immunotypes are distinct from genotypic classification. Similar antigenic cross-reactivity across HIV-1 genotypes has been observed for epitopes from the C5 region of gp120 and for epitopes from the cluster I region of gp41 (Nyambi et al. (2000) J. Virol., 74:10670-10680).

All references cited in the above list of examples are incorporated by reference in their entirety herein.

The first method of the present invention includes the step of providing multiple immunogen preparations derived from the at least one antigenic variation. “Multiple immunogen preparations” encompass the following: (i) where immunotypic variation is due to at least one variation in a single antigen (for example, where the antigen is a peptide epitope that occurs in more than one amino acid sequences), at least one immunogen preparation can be prepared for each of the antigenic variations; (ii) where immunotypic variation is due to variation in more than one antigen (for example, where immunotypic variation is due to variation in two discrete epitopes, proteins, or other antigens), at least one immunogen preparation can be prepared for the antigenic variations. Preferably at least one immunogen preparation is prepared for a majority of the antigenic variations of interest, and more preferably at least one immunogen preparation is prepared for each of the antigenic variations of interest.

Immunogen preparations may be prepared and used as is known in the art (see for example, “Antibodies: A Laboratory Manual”, E. Harlow and D. Lane, editors, Cold Spring Harbor Laboratory, 1988, 726 pp; “Monoclonal Antibodies: A Practical Approach”, P. Shepherd and C. Dean, editors, Oxford University Press, 2000, 479 pp.; and “Chicken Egg Yolk Antibodies, Production and Application: IgY-Technology (Springer Lab Manual)”, by R. Schade et al., editors, Springer-Verlag, 2001, 255 pp., which are incorporated by reference in their entirety herein). An immunogen preparation is derived from an antigenic variation, and can include at least one intact antigenic variation (for example, an intact protein), or at least one portion (for example, a hapten, or a discrete epitope) of an antigenic variation.

The immunogen can include use of at least one naturally occurring, synthesized, combinatorially synthesized, or biologically or recombinantly produced homologue of the naturally occurring antigenic variation. In one embodiment, the immunogen can include multiple sequential immunogenic portions (such as immunogenic peptide or carbohydrate sequences), wherein each immunogenic portion is contiguous with an adjacent immunogenic portion. In another embodiment, the immunogen can include multiple non-sequential immunogenic portions (such as immunogenic peptide or carbohydrate sequences) linked covalently by spacer or linker portions, or linked non-covalently. In a non-limiting example, where the antigen is a protein or peptide, the immunogen can include a recombinant homologue, a mimotope homologue of an epitope derived from the antigen (see, for example, Roccasecca et al. (2001) Mol. Immunol., 38:485-492; Frasca et al. (2003) Hepatology, 38:653-663), or antibodies or antibody fragments that are anti-idiotypic to at least one antibody or antibody fragment capable of binding to the antigen (see, for example, Vogel et al. (2000) J. Mol. Biol., 298:729-735). In another non-limiting example, where the antigen includes a carbohydrate, the immunogen can include a mimotope homologue of an epitope derived from the antigen (see, for example, Harris et al (2000) Infect. Immun., 68-5778-5784; Monzavi-Karbassi et al. (2003) Vaccine, 21:753-760), or antibodies or antibody fragments that are anti-idiotypic to at least one antibody or antibody fragment capable of binding to the antigen (see, for example, Hutchins et al. (1996) Mol. Immunol., 33:503-510; Sacks et al. (1985) J. Immunol., 135:4155-4159). All references cited in this paragraph are incorporated by reference in their entirety herein.

The immunogen can include chemical, physical, biological, or enzymatic modification. Such modification can include, but is not limited to, treatment of the immunogen with a chemical reagent or with an enzyme, heating or cooling, chemical or physical ionization, oxidation or reduction, or incorporation into a liposome, emulsion, or micellar preparation. Where an immunogen is derived from a membrane-associated molecule (such as a membrane-bound or membrane-attached protein, receptor, or carbohydrate), the immunogen can include the molecule associated with the membrane or substantially or completely isolated from the membrane, and can include only a portion or portions of the molecule whether surface-exposed or buried in the membrane. The immunogen can be modified by attachment of a label or a chemical moiety, by a change in the immunogen's configuration (for example, by the introduction of an intramolecular bond), or by crosslinking to a carrier molecule (such as, but not limited to, bovine serum albumin, ovalbumin, or keyhole limpet hemocyanin) (see, for example, R. P. Haugland, “Handbook of Fluorescent Probes and Research Products”, 9^{th} edition, J. Gregory (editor), Molecular Probes, Inc., Eugene, Oreg., USA, 2002, 966 pp.; Seitz and Kohler (2001), Chemistry, 7:3911-3925; Pierce Technical Handbook, Pierce Biotechnology, Inc., 1994, Rockford, Ill.; and Pierce 2003-2004 Applications Handbook and Catalog, Pierce Biotechnology, Inc., 2003, Rockford, Ill., which are incorporated by reference in their entirety herein). The immunogen can be modified by expression or attachment of the immunogen to a scaffold, such as, but not limited to, expression or attachment of one or more epitopes on a polypeptide framework or other molecular framework (see, for example, Skerra, 2000, J. Mol. Recognit., 13:167; Kamb, et al., U.S. Pat. No. 6,025,485; Christmann et al., 1999, Protein Eng., 12:797; Abedi et al., 1998, Nucleic Acids Res., 26:623; and Peelle et al., 2001, J. Protein Chem., 20:507, which are incorporated by reference in their entirety herein). The immunogen can be expressed naturally or recombinantly on the surface of an organism or a particle, for example, by expression of one or more epitopes on a cell (see, for example, Goldberg et al. (1992), Proc. Nat'l. Acad. Sci. USA, 89:10716-10720) or on a virus (see, for example, Staczek et al. (1998) Infect. Immun., 66:3990-3994, and Brennan et al. (1999), Microbiology, 145:211-220, which are incorporated by reference in their entirety herein).

The first method of the present invention also includes the step of immunizing at least one animal with the multiple immunogen preparations. Suitable animals for immunization are vertebrates capable of producing antibodies suitable for the first method of the invention. Preferred animals for immunization include, but are not limited to, chickens, mice, rats, rabbits, sheep, goats, cattle, horses, non-human primates, and humans. The at least one animal can be immunized with one, or with more than one, of the multiple immunogen preparations. Where the at least one animal includes two or more animals, each animal can be immunized with the same or with different immunogen preparations or combinations of immunogen preparations. Where the at least one animal is immunized with more than one of the multiple immunogen preparations, administration of the immunogen preparations can be carried out simultaneously or sequentially.

Non-limiting examples of immunogen administration include the following: The immunogen can be administered as a substantially unmodified immunogen preparation, for example, as a linear or non-linear synthetic or recombinant peptide immunogen, as an intact globular protein, as a protein associated with membrane components, or as a whole or lysed cell. An immunogen preparation can optionally include an adjuvant, such as, but not limited to, Freund's complete or incomplete adjuvant, inorganic or metal salts (such as aluminum salts), and an immunostimulatory molecule (see, for example, Horner et al. (1998) Cell. Immunol., 190:77-82). The immunogen can be administered by immunizing with a molecule including a nucleic acid that encodes the immunogen of interest for in vivo expression (see, for example, Prinz et al. (2003) Immunol, 110:242-249). An immunogen preparation can optionally include a co-stimulatory factor (Frauwirth and Thompson, 2002, J. Clin. Invest., 109:295). Suitable co-stimulatory factors include, for example, cytokines, mitogens, antibodies, antigen-presenting cells (Mayordomo et al., 1997, Stem Cells, 15:94), and peptides derived from a helper T-lymphocyte epitope foreign to the immunized animal. Co-stimulatory factors may be delivered simultaneously with the immunogen used for immunization, for example as a fusion with the immunogen, or separately, for example, as a peptide encoded by a nucleic acid molecule. All references cited in this paragraph are incorporated by reference in their entirety herein.

The first method of the present invention further includes the step of selecting at least one antibody from the at least one immunized animal. The selected at least one antibody of the first method of the present invention can be any suitable antibody, including, but not limited to, polyclonal antibodies, monoclonal antibodies, and antibody fragments, the preparation of which is known in the art (see, for example, “Antibodies: A Laboratory Manual”, E. Harlow and D. Lane, editors, Cold Spring Harbor Laboratory, 1988, 726 pp; “Monoclonal Antibodies: A Practical Approach”, P. Shepherd and C. Dean, editors, Oxford University Press, 2000, 479 pp.; and “Chicken Egg Yolk Antibodies, Production and Application: IgY-Technology (Springer Lab Manual)”, by R. Schade et al., editors, Springer-Verlag, 2001, 255 pp., which are incorporated by reference in their entirety herein). Antibodies may be natural, modified, or recombinant. Antibody fragments include, but are not limited to, F(ab′)₂ fragments, Fab′ fragments, Fab fragments, Fv fragments, and complementarity determining regions (CDRs). Recombinant antibodies include, but are not limited to, single-chain antibody variable region fragments (scFv), miniantibodies (Müller et al. (1998) FEBS Lett., 432:45-49), antibody fusion proteins, and the like (see, for example, “Antibody Engineering”, R. Kontermann and S. Dübel, editors, Springer-Verlag, Berlin Heidelberg, 790 pp.). Antibodies can be monovalent or polyvalent, such as divalent (Plückthun and Pack (1997) Immunotechnology, 3:83-105; Pack et al. (1995) J. Mol. Biol., 246:28-34). Antibodies can be monospecific or polyspecific, such as bispecific (Müller et al. (1998) FEBS Lett., 432:45-49). All references cited in this paragraph are incorporated by reference in their entirety herein.

The at least one antibody selected according to the first method of the invention preferably is of improved cross-reactivity for the infectious organism of interest, relative to an antibody from an animal immunized with only a single immunogen derived from the infectious organism. By cross-reactivity is meant immunologically reactive across more than one immunotype of the infectious organism of interest. The selected at least one antibody of improved cross-reactivity is preferably cross-reactive against a majority of the immunotypes of interest, more preferably cross-reactive against a substantial majority of the immunotypes of interest, and most preferably cross-reactive against all or substantially all of the immunotypes of interest. In addition, the selected at least one antibody of improved cross-reactivity preferably does not non-specifically bind to substances (such as other organisms) in the sample other than the infectious organism of interest. Immunological characterization of the at least one antibody's qualities (such as its specificity and affinity for its intended antigen and its binding kinetics) may make use of any suitable method, including, but not limited to, dot blot assays, ELISA assays, competitive immunoassays, displacement immunoassays, radioimmunometric assays, agglutination assays, surface plasmon resonance measurements, and combinations thereof. The at least one antibody can in some cases be a single antibody, for example, a single isolated polyclonal antibody preparation (such as a single polyclonal antibody preparation that includes a mixture of serum immunoglobulins or immunoglobulin derivatives prepared by a specific affinity purification protocol) or a monoclonal antibody produced by a single hybridoma clone. The at least one antibody can in some cases be a few antibodies, such as from about 2 to about 10 isolated polyclonal antibody preparations or such as monoclonal antibodies produced from about 2 to about 10 separate hybridoma clones. The at least one antibody can in some cases be several antibodies, such as more than about 10 isolated polyclonal antibody preparations or such as monoclonal antibodies produced from more than about 10 separate hybridoma clones.

Selection of the at least one antibody of improved cross-reactivity can include any suitable selection method, including, but not limited to, salt precipitation, size selection by filtration or centrifugation, affinity separation techniques (for example, affinity chromatography, affinity precipitation), ion-exchange and other chromatographic methods, ligand exchange, thiophilic adsorption, purification by use of immunoglobulin binding substances (for example, protein A, protein G, protein L, jacalin and other lectins, and mannan binding protein). Selection can include use of panning techniques (see, for example, Coomber (2001) Methods Mol. Biol., 178:133-145; Zhou et al. (2002) Proc. Natl. Acad. Sci. USA, 99:5241-5246; Fehrsen and du Plessis (1999) Immunotechnology, 4:175-184; Deng et al. (1994) J. Biol. Chem., 269:9533-9538; Burioni et al. (1998) Res. Virol., 149:327-330; Boel et al. (1998) Infect. Immun., 66:83-88; and Parsons et al. (1996) Protein Eng., 9:1043-1049, which are incorporated by reference in their entirety herein).

Selection of the at least one antibody of improved cross-reactivity can include the use of directed evolution of an antibody or antibody fragment (see, for example, Barbas et al. (1994), Proc. Nat'l. Acad. Sci. USA., 91:3809-3813, which is incorporated by reference in its entirety herein). Selection of the at least one antibody of improved cross-reactivity can include the use of directed evolution of an immunogen, such as of an epitope or peptide in a recombinant expression system or in a combinatorial system. Suitable immunogen directed evolution techniques include, but are not limited to, displaying on a polypeptide (Kamb, et al., U.S. Pat. No. 6,025,485; Christmann et al., 1999, Protein Eng., 12:797; Abedi et al., 1998, Nucleic Acids Res., 26:623; Peelle et al., 2001, J Protein Chem., 20:507), a phage (He, 1999, J. Immunol. Methods, 231:105; Smith, 1985, Science, 228:1315), a ribosome (Schaffitzel et al., 1999, J. Immunol. Methods, 231:119; Roberts, 1999, Curr. Opin. Chem. Biol., 3:268), an mRNA (Wilson et al., 2001, Proc. Natl. Acad. Sci., 98:3750), or a yeast cell surface (Yeung and Wittrup, 2002, Biotechnol. Prog., 18:212; Shusta et al., 1999, J. Mol. Biol., 292:949), a bacterial cell surface (Leenhouts et al., 1999, Antonie Van Leeuwenhoek, 76:367; Christmann et al., 2001, J. Immunol. Methods, 257:163), or a bacterial spore surface (Wittrup, 2001, Curr. Opin. Biotechnol., 12:395; Boder and Wittrup, 1998, Biotechnol. Prog., 14:55). All references cited in this paragraph are incorporated by reference in their entirety herein.

The first method of the present invention can further include the optional step of improving the affinity of the selected antibodies. Antibody affinity can be improved by any suitable method, such as, but not limited to affinity separation and directed evolution techniques. Affinity separation can make use of the antibodies' affinity for original antigen, original immunogen, modified antigen, modified immunogen, or mimics of antigen or immunogen such as mimotopes (see, for example, Smith et al. (2002) J. Chromatogr. B Analyt. Technol. Biomed. Life Sci., 766:13-26; Murray et al. (1997) J. Chromatogr. A, 782:49-54). Directed evolution techniques include display methods, such as, but not limited to, displaying on phages, ribosomes, and mRNA. See, for example, Schaffitzel et al., (1999) J. Immunol. Methods, 231:119-135; Proba et at (1998) 275:245-253; He and Taussig (1997) Nucleic Acids Res., 25:5132-5134; Xu et at (2002) Chem. Biol, 9:933-942; Xu et at (2003) Chem. Biol., 10:91-92; Boder et at (2000) Proc. Nat'l Acad. Sci. USA, 97:10701-10705; Scheir et at (1996) J. Mol. Biol., 263:551-567; Barbas and Burton (1996) Trends Biotechnol., 14:230-234; and Crameri et at (1996) Nature Med., 2:100-102, which are incorporated by reference in their entirety herein.

Antibodies generated by the first method of the invention may be used for any purpose desired, including immunochemical diagnostics, immunochemical affinity isolation of antigens, immunochemical labelling, immunochemical studies of antigen variation, evolution or epidemiology, in studies of vaccine development, as prophylaxis or treatment of an infectious disease in a subject, and the like. One preferred use of antibodies generated by the first method of the invention is immunochemical diagnosis of infectious disease states in human subjects.

In one non-limiting example of the first method of the invention, the infectious organism of interest is non-typeable Haemophilus influenzae (NTHi), which can be immunotyped based on antigenic differences of the major outer membrane protein, OMP2, a surface antigen (Haase et at (1994) Infect. Immun., 62:3712-3722; Groeneveld et al. (1989) Infect. Immun., 57:3038-3044). In some cases, the antigenic differences may be due to amino acid sequence variations of discrete surface-exposed OMP2 loops; such variations can result in distinguishable OMP2 epitopes found on a single loop or in a combination of loops. Antigenic differences may also be due to amino acid variations in portions of the OMP2 molecule that are at least substantially associated with the membrane.

The multiple immunogen preparations can be derived from OMP2 by any suitable technique. Intact, broken, lysed, or extracted non-typeable Haemophilus influenzae cells or cellular components (such as cell membranes) can be used as OMP2 immunogens. OMP2 protein, associated with cell membrane or cell membrane components, or isolated completely or substantially from the cell membrane, can be used intact or in fragments or in modified form as OMP2 immunogens. OMP2 immunogen preparations can include one or more protein fragments, such as, but not limited to surface-exposed sequences (for example, the variable outer loops), membrane-buried sequences (for example, trans-membrane consensus sequences), cytosolic membrane-associated sequences, and combinations thereof. Other suitable OMP2 immunogen preparations can include linear or non-linear natural, recombinant, or synthetic peptides derived from OPM2, recombinant proteins or fusion proteins derived from OMP2, mimotopes that mimic epitopes of OMP2, and antibodies or antibody fragments that are anti-idiotypic to at least one antibody capable of binding to OMP2.

Any suitable animal can be immunized with the multiple OMP2 immunogen preparations, using an immunization administration procedure appropriate to the species of animal, the multiple immunogen preparations, and the intended antibody. In a non-limiting example, where the multiple immunogen preparations include substantially intact OMP2 protein purified from immunologically distinct non-typeable Haemophilus influenzae strains, and rabbit polyclonal cross-reactive anti-OMP2 antibodies are desired, non-limiting examples of suitable immunization procedures include: (i) immunizing groups of one or more rabbits (for example, groups of one to ten rabbits), wherein each animal in a group is immunized with the identical combination of immunogen preparations, each group is immunized with a different combination of immunogen preparations, and each immunogen preparation is used at least once; (ii) immunizing at least one, and preferably several, rabbits, each individual rabbit being immunized with all of the multiple immunogen preparations; and (iii) immunizing groups of one or more rabbits (for example, groups of one to ten rabbits), wherein each animal in a group is immunized with one of the immunogen preparations, and wherein each immunogen preparation is used at least once. In other non-limiting examples, immunization procedures similar to the preceding may use multiple immunogen preparations that include linear or non-linear peptides derived from OMP2 protein from immunologically distinct non-typeable Haemophilus influenzae strains, or recombinant proteins or fusion proteins derived from such OMP2 proteins, or mimotopes that mimic epitopes of such OMP2 proteins.

II. Method for Detecting an Infectious Organism

The present invention also includes a method to detect an infectious organism that exists in more than one immunotype, wherein the immunotypes are due to at least one antigenic variation, the method including the steps of: (a) providing a sample suspected of containing the at least one antigenic variation; (b) providing at least one antibody generated by the first method of the present invention as described above under the heading “I. METHOD FOR GENERATING ANTIBODIES OF IMPROVED CROSS-REACTIVITY”; (c) contacting the sample with the at least one antibody, under conditions that allow the at least one antibody to bind to and form a complex with the at least one antigenic variation; and (d) detecting the complex, wherein the detection is positive if concentration of the infectious organism in the sample is greater than or equal to than a reference concentration, and the detection is negative if concentration of the infectious organism in the sample is less than the reference concentration. When used with respect to the second method of the present invention, the terms and concepts “immunotype”, “immunotypic determination”, “immunotypic variation”, “antigenic variation”, “antigen”, “immunogen”, “immunogen preparation”, “immunogen modification”, “immunogen administration”, and “cross-reactivity” are as described or defined above under the heading “I. METHOD FOR GENERATING ANTIBODIES OF IMPROVED CROSS-REACTIVITY”.

The second method of the present invention may be applied to any infectious organism (or combination of infectious organisms) that exists in more than one immunotype, including bacteria (including mycoplasmas), viruses, and eukaryotic pathogens (including pathogenic fungi and protozoans). Immunotypic variation is due to at least one antigenic variation, that is to say, variation found in at least one antigen of the infectious organism. Thus, the at least one antigenic variation results in at least two distinguishable immunotypes. More preferably, the second method of the present invention may be applied to pathogens that infect vertebrates, invertebrates, or plants. Most preferably, the method may be applied to pathogens that infect humans, mammals, birds, fish, insects, or plants. A non-limiting selection of human pathogens of particular interest include Haemophilus influenzae (including non-typeable Haemophilus influenzae), Streptococcus pneumoniae, Moraxella catarrhalis, Neisseria gonorrhoea, Neisseria meningitidis, Pseudomonas aeruginosa, Staphylococcus aureus, Listeria monocytogenes, Chlamydia trachomatis, Chlamydia pneumoniae, Chiamydia psittaci, Corynebacterium diptheriae, Mycobacterium tuberculosis, Mycoplasma pneumoniae, Bordetella pertusssis, Legionella species, Pneumocystis carinii, Nocardia species, Pasteurella multocida, Klebsiella rhinoscieromatis, Francisella tularensis, Bacillus anthracis, Yersinia pestis, Pseudomonas pseudomallei, Coxiella burnetti, Brucella species, Salmonella species, Histoplama capsulatum, Coccidiodes immitis, Cryptococcus neoformans, Blastomyces dermatidis, influenza viruses, human immunodeficiency viruses, rhinoviruses, respiratory syncytial viruses, adenoviruses, enteroviruses, herpes viruses, parainfluenza viruses, varicella-zoster viruses, and eukaryotic pathogens. The method may be applied particularly when it is of interest to detect or monitor the presence or concentrations of the infectious organism of interest, regardless of the immunotypic composition of that infectious organism's population.

The second method of the present invention includes the step of providing a sample suspected of containing the at least one antigenic variation. A suitable sample is one suspected to contain at least one antigenic variation of interest. Antigens can include, but are not limited to, peptides, proteins, carbohydrates, lipids, glycoproteins, glycolipids, lipoproteins, lipopolysaccharides, nucleic acids, and combinations thereof. Antigens can include intact molecules, fragments of molecules, and multi-molecular complexes. Antigens can be antigens as they naturally occur, or antigens that require chemical, physical, biological, or enzymatic modification to, for example, become available for interaction with an antibody. The antigen can be modified, for example, by physical or chemical modification, including, but not limited to, treatment with chemical reagents or enzymes, oxidation or reduction, labelling with a detectable label, and covalent or non-covalent attachment of the epitope to a separate moiety, molecule, molecular structure, or surface.

The sample may be any sample of interest, including, but not limited to, samples of entirely natural origin, of entirely non-natural origin (such as of synthetic origin), or a combination of natural and non-natural origins. Suitable samples include, but are not limited to, clinical samples, pathological samples, biological samples, environmental samples, and experimental research samples. Samples can include cells, tissues, or organs, any or all of which can be can be intact or disrupted or lysed or otherwise modified. Samples can include biological materials (such as, but not limited to, blood, serum, plasma, urine, feces, semen, mucous, discharges, and cerebrospinal fluid), washes, aspirates, or swabs (such as oropharyngeal or nasopharyngeal washes, aspirates, or swabs), tissue samples, or a combination thereof A sample may be an extract made from biological materials, such as from prokaryotes, bacteria, eukaryotes, plants, fungi, multicellular organisms or animals, invertebrates, vertebrates, mammals, non-human mammals, and humans. A sample may be an extract made from whole organisms or portions of organisms, cells, organs, tissues, fluids, whole cultures or portions of cultures, or environmental samples or portions thereof. Samples can include extraction fluids, buffers, solvents, or other chemical or biological reagents as necessary. In some cases, a suitable sample can be one suspected to contain the infectious organism of interest in any suitable condition, such as, but not limited to, whole or intact organisms, disrupted or lysed organisms, and organisms modified by physical, chemical, biological, or enzymatic treatment or combination of treatments. In some cases, a suitable sample can be one suspected to contain the antigenic variation without substantial presence of the infectious organism of interest, for example, where the sample includes substantially isolated cellular components (such as cell membranes or cell walls) or substantially isolated antigens (such as isolated or purified proteins or carbohydrates). A sample can include a crude or semi-purified or purified antigen, or a mimic of the antigen, such as a mimotope. A sample may need minimal preparation (for example, collection into a suitable container) for use in a method of the present invention, or more extensive preparation (such as, but not limited to: treatment with one or more reagents; removal, inactivation, or blocking of undesirable material, such as contaminants, undesired components, or endogenous enzymes; filtration, size selection, or affinity purification; tissue or cell fixation, embedding, or sectioning; tissue permeabilization or cell lysis; or concentration or dilution).

The second method of the present invention also includes the step of providing at least one antibody generated by the first method of the present invention as described above under the heading “I. METHOD FOR GENERATING ANTIBODIES OF IMPROVED CROSS-REACTIVITY”. The at least one antibody preferably is of improved cross-reactivity for the infectious organism of interest, relative to an antibody from an animal immunized with only a single immunogen derived from the infectious organism. The at least one antibody is preferably cross-reactive against a majority of the immunotypes of interest, more preferably cross-reactive against a substantial majority of the immunotypes of interest, and most preferably cross-reactive against all or substantially all of the immunotypes of interest.

The at least one antibody can include at least one polyclonal antibody, at least one monoclonal antibody, at least one antibody fragment, or any combination thereof. Suitable antibodies can be natural, modified, or recombinant, and can include F(ab′)₂ fragments, Fab′ fragments, Fab fragments, Fv fragments, complementarity determining regions (CDRs), single-chain antibody variable region fragments (scFv), miniantibodies, antibody fusion proteins, and engineered mimics of antibodies. Antibodies can be monovalent or polyvalent, and can be monospecific or polyspecific. The at least one antibody can be a single antibody, a few antibodies, or several antibodies. The at least one antibody may be capable of binding to a mimotope, such as a peptide, that mimics an epitope naturally derived from the infectious organism (see, for example, Kieber-Emmons (1998) Immunol. Res., 17:95-108; Shin et al. (2001) Infect. Immun., 69:3335-3342; Beenhouwer et al. (2002) J. Immunol., 169:6992-6999; Hou and Gu (2003) J. Immunol., 170:4373-4379; and Tang et al. (2003) Clin. Diagn. Lab. Immunol., 10:1078-1084, which are incorporated by reference in their entirety herein).

The at least one antibody can optionally include a functional group (such as a chemically reactive moiety or cross-linking moiety) or a detectable label; methods to introduce such functional groups or detectable labels are known in the art (see, for example, R. P. Haugland, “Handbook of Fluorescent Probes and Research Products”, 9^{th} edition, J. Gregory (editor), Molecular Probes, Inc., Eugene, Oreg., USA, 2002, 966 pp.; Seitz and Kohler (2001), Chemistry, 7:3911-3925; Pierce Technical Handbook, Pierce Biotechnology, Inc., 1994, Rockford, Ill.; and Pierce 2003-2004 Applications Handbook and Catalog, Pierce Biotechnology, Inc., 2003, Rockford, Ill., which are incorporated by reference in their entirety herein). The at least one antibody can be used in more than one form or type, for example, in a sandwich assay that involves one form of the at least one antibody to immobilize the antigen and at least one detectably labelled form of the at least one antibody that binds the same antigen.

The at least one antibody may be free in solution, or may be temporarily or permanently immobilized, directly or indirectly, onto a separate moiety, molecule, molecular structure, matrix, or surface. In one non-limiting example of direct immobilization, the at least one antibody can be temporarily immobilized by drying or otherwise transiently binding onto a surface or into a matrix, wherein addition of a fluid can cause the at least one antibody to become mobile. In another non-limiting example of direct immobilization, the at least one antibody can be permanently immobilized by covalent or non-covalent attachment to a surface, such as to a membrane, microplate well, tube, chip, or slide. In a non-limiting example of indirect immobilization, the at least one antibody can be affixed by covalent or non-covalent means (such as by passive adsorption or avidin/biotin binding) to particles (for example, beads or fibers or particles of latex, gold or other metals, or magnetic or paramagnetic materials), and the antibody-bearing particles temporarily or permanently immobilized onto a surface or within a matrix. In another non-limiting example of indirect immobilization, the at least one antibody can be affixed using a linking moiety, such as a cross-linking molecule or a multivalent molecule (such as avidin), to a separate moiety, molecule, molecular structure, matrix, or surface.

The second method of the present invention also includes the step of contacting the sample with the at least one antibody, under conditions that allow the at least one antibody to bind to and form a complex with the at least one antigenic variation. Preferably, the at least one antibody binds to the at least one antigenic variation to form a complex of sufficient stability to be detected. The at least one antibody binds preferably to a majority of the antigenic variations that give rise to the immunotypes of interest, more preferably to a substantial majority of the antigenic variations that give rise to the immunotypes of interest, and most preferably to all or substantially all of the antigenic variations that give rise to the immunotypes of interest. Also preferably, the at least one antibody binds to the at least one antigenic variation with sufficient specificity to give minimal or no binding between the at least one antibody and an antigen other than that in which variation gives rise to the immunotypes of interest.

The at least one antigenic variation may be modified, such as modified before the binding occurs. Modification of an antigen may be of interest, for example, where modification improves the binding affinity or selectivity of the at least one antibody to the at least one antigenic variation, or where modification is used to separate the at least one antigenic variation from other substances in a sample. Where the antigen to which the at least one antibody is intended to bind is a modified antigen (for example, an antigen modified by physical or chemical treatment), the at least one antibody is preferably capable of specifically binding to the modified antigen. In one non-limiting example, the antigen may be modified by introduction of a chemical functional group (such as a thiol, hydroxyl, aldehyde, amine, carboxylate, or other reactive group), cross-linking agent, or other moiety (for example, biotin or avidin), which permits the antigen to be immobilized by appropriate chemical reaction to a matrix or surface. In another non-limiting example, the antigen may be modified by treatment with one or more chemical or enzymatic reagents (such as, but not limited to, detergents, solvents, chaotropes, lipases, proteases, glycosidases, and the like) to improve antibody-antigen binding.

The second method of the present invention also includes the step of detecting the complex formed between the at least one antibody and the at least one antigenic variation, wherein the detection is positive if concentration of the infectious organism in the sample is greater than or equal to than a reference concentration, and the detection is negative if concentration of the infectious organism in the sample is less than the reference concentration. Detection of the complex can be direct, such as by detection of a label on the at least one antibody. Alternatively, detection of the complex can be indirect, by any suitable means, including, but not limited to, the use of a secondary antibody, such as a secondary antibody bearing a detectable label. Useful detectable labels include, but are not limited to, fluorophores, luminophores, members of resonance energy transfer pairs, lanthanides, dyes, pigments, radioactive isotopes, magnetic labels, spin labels, heavy atoms, metals, particles (such as gold particles or magnetic particles), and enzymes.

Detection of the complex is positive if the concentration of the infectious organism in the sample is greater than or equal to a reference concentration. Conversely, detection of the complex is negative if the concentration of the infectious organism in the sample is less than the reference concentration. The reference concentration selected for a given infectious organism depends on several factors, including, but not limited to, the infectious organism, the nature of the at least one antibody and of the at least one antigenic variation, the type of sample, and the source of the sample (which may be a human subject, for example, an adult or a child). Reference concentrations can be established by routine testing. Detection can be linear (such as spectrophotometric measurement of product formation by an enzymatic reaction) or non-linear (such as visual detection of a gold label). Detection is optionally at least semi-quantitative, for example, judged to be greater than or equal to, or less than, a reference value, or for example, judged to be much greater than, moderately greater than or equal to, or less than, a reference value. Detection can be optionally quantitative, wherein a positive detection signal can be correlated to a range of concentrations of the infectious organism.

In some cases, where the presence or absolute absence of an infectious organism in a sample is of interest, the reference value can be zero. However, the reference value can be any suitable value or range of values, depending on the purpose of the analysis. In one embodiment of the invention where the diseased or not diseased condition of a subject is of interest, a desirable reference concentration preferably yields a positive predictive value (that is to say, the probability that the subject who generated the sample giving a positive detection result is diseased by the infectious organism) of at least about 80%, more preferably of at least about 90%, and most preferably of at least about 95%. In such an embodiment, a desirable reference concentration preferably yields a negative predictive value (that is to say, the probability that the subject who generated the sample giving a negative detection result is not diseased by the infectious organism) of at least about 80%, more preferably of at least about 90%, and most preferably of at least about 95%. In some cases, certain such embodiments of the invention can give a semi-quantitative estimate of the concentrations of the infectious organism, based on the strength of the positive detection signal. In other cases, the positive detection signal can be quantitatively correlated to a range of concentrations of the infectious organism above the reference value.

In some cases, a sample may be from a subject who is a “carrier” of an infectious organism, that is to say, otherwise healthy but colonized, generally at relatively lower concentrations, by the infectious organism, where a relatively higher concentration of the infectious organism is associated with symptoms of a disease caused by that organism. In such a case, a desirable reference concentration may be a concentration below which a sample from a subject who either is not colonized by the infectious organism in question, or who is colonized by the infectious organism but otherwise healthy, gives a negative detection result. This same reference concentration is preferably a concentration at or above which a sample from a subject who is colonized and diseased by the infectious organism gives a positive detection result. Thus, in one embodiment of the invention, a positive detection result indicates that the sample is from a subject who is at least colonized by the infectious organism of interest, or is colonized and diseased by that organism. In one alternative embodiment of the invention, a negative detection result preferably indicates that the sample is from a subject who is not colonized by the infectious organism of interest to a level associated with a disease caused by that infectious organism.

The second method of the present invention may optionally be applied to more than one antigenic variation (from a single or from more than one infectious organism), or to more than one infectious organism, simultaneously or in parallel. In some cases, it may be of interest to detect the presence or levels of one or more infectious organisms that are capable of causing similar symptoms in order to distinguish cause of disease and to determine an appropriate course of therapy. In one non-limiting example, the method may be used to distinguish different strains of a single species of infectious organism, such as where the different strains vary in their pathogenicity or in their susceptibility to a given drug or antibiologic. In another non-limiting example, it may be of interest to distinguish between species or strains of viral pathogens (see, for example, Mackie (2003), Paediatr. Respir. Rev., 4:84-90) that can cause similar symptoms of respiratory disease, or to distinguish viral pathogens from bacterial or fungal pathogens that can cause similar symptoms of respiratory or gastrointestinal or central nervous system disease (see, for example, Murphy (2003) Curr. Opin. Infect. Dis., 16:129-134; Tan (2002) Semin. Respir. Infect., 17:3-9; Heikkinen and Chonmaitree (2003) Clin. Microbiol. Rev., 16:230-241; Leclerc et al. (2002) Crit. Rev. Microbiol., 28:371-409; and Sferra and Pacini (1988) Pediatr. Infect. Dis. J, 7:552-556), and thus to help determine the appropriate course of therapy, such as the appropriate antivirals or antibacterials (Fendrick et al. (2001) Clin. Ther., 23:1683-1708). In another non-limiting example, the second method of the present invention can be useful in the control of infectious disease, for example, to determine the need for isolation of a diseased individual or individual in order to prevent outbreaks or epidemics. All references cited in this paragraph are incorporated by reference in their entirety herein.

III. Kit for Detecting an Infectious Organism

The present invention also includes a kit to use the second method of the invention for detecting an infectious organism that exists in more than one immunotype. Suitable kits can be designed for convenience in performing the method, according to the assay used. Suitable assays include, but are not limited to, dipstick or test strip assays, flow-through assays, chromatographic assays, affinity separation assays, lateral flow assays, latex agglutination assays, radioimmunometric assays, enzyme-linked immunosorbent assays, fluorescence assays, and luminescence assays. Assays can be run in any suitable format, including, but not limited to, membranes, filters, microtiter plates, tubes, chips, slides, and flow-through chambers. Preferably, the assay is rapid, most preferably sufficiently rapid to produce results within a relatively brief period of time, such as the time of a subject's consultation with a physician or other health-care provider. In some cases, the kits and assays can be designed to be run on a single or on few samples. In other cases, the kits and assays can be designed to be run on many samples, for example, in a multiple-well format or in a high-throughput screen.

Kits can include, in addition to a means for performing the assay, means for collecting and appropriately treating a sample (such as a swab, a needle and syringe, a Vacutainer® or a Monovette®, a means to aspirate a sample, wash solutions or buffers, cell dissociation or lysis reagents, chemical or enzymatic reagents, filters, centrifuge tubes, and the like). Kits can include standards, such as semi-quantitative or quantitative standards. Kits can include controls, such as positive or negative controls or both. Kits can include materials (such as gloves and other personal safety equipment, biohazard disposal containers, or decontamination materials) that aid in the safe handling of potentially hazardous samples. Kits can include instructions for the use of the kit, for trouble-shooting, or for interpretation of results; these may be, for example, instructions in the form of a brochure, leaflet, pamphlet, booklet, or audiovisual materials.

IV. Another Method for Generating Antibodies of Improved Cross-Reactivity

The present invention includes another method for generating a selection of antibodies having improved cross-reactivity for an infectious organism that exists in more than one immunotype, wherein the immunotypes are due to at least one antigenic variation, the method including the steps of: (a) providing multiple immunogen preparations derived from the at least one antigenic variation; (b) immunizing multiple groups of animals, wherein each of the groups comprises at least one animal, and wherein each of the animals is immunized with a single immunogen preparation; (c) selecting at least one antibody from each of the groups; and (d) combining the selected antibodies, wherein the combination of selected antibodies is of improved cross-reactivity for the infectious organism. When used with respect to the fourth method of the present invention, the terms and concepts “immunotype”, “immunotypic determination”, “immunotypic variation”, “antigenic variation”, “antigen”, “immunogen”, “immunogen preparation”, “immunogen modification”, “immunogen administration”, and “cross-reactivity” are as described or defined above under the heading “I. METHOD FOR GENERATING ANTIBODIES OF IMPROVED CROSS-REACTIVITY”. The fourth method of the present invention may be applied to any infectious organism that exists in more than one immunotype, including bacteria (including mycoplasmas), viruses, and eukaryotic pathogens, most preferably pathogens that infect humans, mammals, birds, fish, insects, or plants.

The fourth method of the invention includes the step of providing multiple immunogen preparations derived from the at least one antigenic variation. The multiple immunogen preparations can be prepared and administered as described above under the heading “I. METHOD FOR GENERATING ANTIBODIES OF IMPROVED CROSS-REACTIVITY”.

The fourth method of the present invention also includes the step of immunizing multiple groups of animals, wherein each of the groups comprises at least one animal, and wherein each of the animals is immunized with a single immunogen preparation. Animals that may be immunized include, but are not limited to, chickens, mice, rats, rabbits, sheep, goats, cattle, horses, non-human primates, and human. Where there are a number of multiple immunogen preparations equal to n, preferably n groups of animals and thus at least n animals are immunized.

The fourth method of the present invention also includes the step of selecting at least one antibody from each of the groups. Selecting can employ any selection method as described above under the heading “I. METHOD FOR GENERATING ANTIBODIES OF IMPROVED CROSS-REACTIVITY”. Preferably, but not necessarily, each of the selected at least one antibody from each of the groups is individually of improved cross-reactivity for the infectious organism of interest, relative to an antibody from an animal or animals immunized with only a single immunogen derived from the infectious organism.

The fourth method of the present invention further includes the step of combining the selected antibodies, wherein the combination of selected antibodies is of improved cross-reactivity for the infectious organism. The combination of selected antibodies generated by the fourth method of the invention preferably is of improved cross-reactivity for the infectious organism of interest, relative to antibodies from an animal or animals immunized with only a single immunogen derived from the infectious organism. The combination of selected antibodies is preferably cross-reactive against a majority of the immunotypes of interest, more preferably cross-reactive against a substantial majority of the immunotypes of interest, and most preferably cross-reactive against all or substantially all of the immunotypes of interest.

Combinations of selected antibodies generated by the fourth method of the invention may be used for any purpose desired, including immunochemical diagnostics, immunochemical affinity isolation of antigens, immunochemical labelling, immunochemical studies of antigen variation, evolution or epidemiology, in studies of vaccine development, as prophylaxis or treatment of an infectious disease in a subject, and the like. One preferred use of selections of antibodies generated by the fourth method of the invention is immunochemical diagnosis of infectious disease states in human subjects. The selections of antibodies generated by the fourth method of the present invention may be employed in methods to detect an infectious organism that exists in more than one immunotype, as described above under the heading “II. METHOD FOR DETECTING AN INFECTIOUS ORGANISM”, and in kits for such methods, as described above under the heading, “III. KIT FOR DETECTING AN INFECTIOUS ORGANISMN”.

### EXAMPLES

### Example 1

### Preparation of Immunogens

This example describes a non-limiting embodiment of a method for preparing single or multiple immunogens derived from at least one antigenic variation. In this example, the infectious organism of interest is non-typeable Haemophilus influenzae, and the antigen that gives rise to immunotypic variation is outer membrane protein 2 (OMP2).

Non-typeable Haemophilus influenzae (NTHi) strain numbers 19418, 35540, 43163, 49401, 49766, 51997, and 53600 were purchased from the American Type Culture Collection (ATCC) (Manassas, Va.) and cultured on multiple chocolate agar plates or liquid media (Haemophilus broth, catalogue number M-6534, Sigma-Aldrich, St. Louis, Mo., USA, supplemented with 4.4% glycerol, 0.003% hemin, and 0.001% beta-nicotinamide adenine dinucleotide). Strains of NTHi cells are harvested from 24- to 48-hour plate or liquid cultures, by scraping plates or centrifugation of the liquid culture, respectively. The cells are washed several times with hydroxyethylpiperazine ethanesulfonate (HEPES) buffer (pH 7.4) or other suitable wash buffer, such as phosphate buffered saline (PBS). Cells were used immediately in an extraction procedure or stored as frozen wet pellets for a maximum of 2 weeks at −20 degrees Celsius.

The outer membrane protein, OMP2, was isolated from the 7 non-typeable Haemophilus influenzae (NTHi) strains using a procedure based on Murphy and Bartos (1988) Infect Immun., 56:1084, which is incorporated by reference in its entirety herein. In brief, cell membrane proteins were isolated from other cellular components by successive treatment of cells with detergent buffers and differential EtOH precipitation. NTHi cells were harvested, washed with HEPES buffer (0.01 molar, pH 7.4), and centrifuged. The cell pellets were resuspended in HEPES buffer, transferred to Oak Ridge centrifuge tubes, and centrifuged in a Sorvall SA600 rotor at 8000 rotations per minute for 20 minutes at 4 to 6 degrees Celsius. The supernatant was discarded, and the cell pellets resuspended in less than 1 milliliter of HEPES buffer and transferred to a 50-milliliter Erlenmeyer flask containing a stirring bar. To the flask was added 2 milliliters sodium acetate buffer (pH 4.0) containing 0.001 molar beta-mercaptoethanol and 9 volumes of a solution containing 5% Zwittergent 3-14 (catalogue number 693017, Calbiochem, San Diego, Calif., USA), 0.5 molar calcium chloride, 1 microgram per milliliter pepstatin, 0.4 milligrams per milliliter EDTA, 0.1 milligrams per milliliter Pefabloc SC (Fluka catalogue number 76307, Sigma-Aldrich, St. Louis, Mo., USA). The cells were stirred in this solution for at least one hour at room temperature. Ethanol was added to the cell suspension to a final concentration of 20% ethanol by volume. The suspension was mixed, transferred to Oak Ridge centrifuge tubes, and centrifuged in a Sorval SA600 rotor at 11,000 rotations per minute for 10 minutes at 4 to 6 degrees Celsius, to remove nucleic acids and large cellular material. The pellets were discarded. Ethanol was added to the supernatants to a final concentration of 80% ethanol by volume. The suspension was mixed, transferred to Oak Ridge centrifuge tubes, and centrifuged in a Sorval SA600 rotor at 11,000 rotations per minute for 20 minutes at 4 to 6 degrees Celsius. The supernatants were discarded, and the cell pellets resuspended in 3 to 4 milliliters of Buffer Z (0.05 molar Tris, 0.05% Zwittergent 3-14, 0.01 molar EDTA, pH 8.0) containing 1 microgram per milliliter pepstatin and 0.1 milligrams per milliliter Pefabloc SC. The combined cell suspensions were transferred to an Erlenmeyer flask with a stirring bar and stirred for at least 1 hour at room temperature. The suspension was transferred to Oak Ridge centrifuge tubes and centrifuged in a Sorval SA600 rotor at 9,000 rotations per minute for 10 minutes at 4 to 6 degrees Celsius. The supernatants were pooled and labelled as S1 with the strain number. The cell pellets were resuspended in 1 to 2 milliliters of Buffer Z (0.05 molar Tris, 0.05% Zwittergent 3-14, 0.01 molar EDTA, pH 8.0) containing 1 microgram per milliliter pepstatin and 0.1 milligrams per milliliter Pefabloc SC, and stirred for at least 1 hour at room temperature. The suspension was transferred to Oak Ridge centrifuge tubes and centrifuged in a Sorval SA600 rotor at 9,000 rotations per minute for 10 minutes at 4 to 6 degrees Celsius. The supernatants were pooled and labelled as S2 with the strain number. The OMP2 protein preparations can be stored at −80 degrees Celsius in Buffer Z (0.05 molar Tris, 0.05% Zwittergent 3-14, 0.01 molar EDTA, pH 8.0) with 2.5% sucrose added. Protein preparations thus prepared were characterized by suitable methods (such as SDS-PAGE electrophoretic analysis, ELISA, or HPLC). OMP2 proteins can be further purified from other membrane components (such as other proteins, lipooligosaccharides, and glycoproteins) by ion-exchange and/or size-exclusion chromatography.

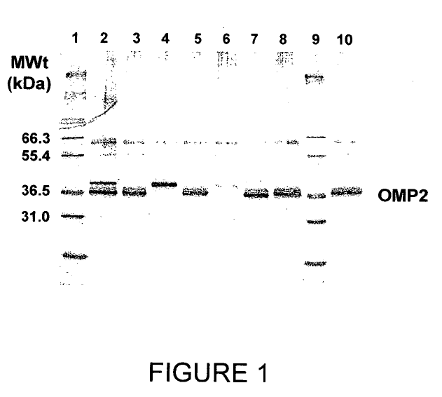

The resulting isolated OMP2 proteins were characterized for purity and banding patterns by denaturing polyacrylamide gel electrophoresis (SDS-PAGE) and silver staining, a representative example of which is depicted in FIG. 1. In accordance with previous work, there were notable differences in strain banding patterns. Molecular weight(s) of the OMP2 band(s) were determined. The average OMP2 molecular weights were calculated from an average of the results from at least 3 protein isolations and electrophoretic analyses, and are given according to ATCC NTHi strain designation in Table 1.

TABLE 1
Calculated Average Molecular Weights
(in kilodaltons) of NTHi OMP2 Isolates

| ATTC | Primary | Secondary |
|---|---|---|
| Strain | Band | Band |
| 51997 | 42.8 | none |
| 49401 | 39.5 | 40.4 |
| 49766 | 41.6 | 40.5 |
| 53600 | 41.0 | 43.3 |
| 43163 | 43.5 | 42.7 |
| 35540 | 43.1 | none |
| 19418 | 40.1 | 44.4 |

### Example 2

### Polyclonal Antibody Generation

This example describes a non-limiting embodiment of a method for preparing polyclonal antibodies against single or multiple immunogens derived from at least one antigenic variation.

Based on their electrophoretic banding diversity, combinations of OMP2 proteins prepared and characterized as described in Example 1 were selected as multiple immunogen preparations for immunization programs. Alternatively, the selection of OMP2 proteins can be based on comparison of OMP2 sequences derived from direct sequencing of OMP2 gene fragments amplified by polymerase chain reaction. Immunogen mixtures of OMP2 proteins for polyclonal antibody production are preferably limited to OMP2 proteins from no more than 3 to 4 NTHi strains per mix to decrease the possibility of one strain being immunodominant. Immunogen mixtures of OMP2 proteins for monoclonal hybridoma production can contain OMP2 proteins from several NTHi strains, including from more than 3 to 4 strains, as screening of hybridoma clones permits selection of strain-specific or cross-reactive clones.

Polyclonal antiserum to strain mixtures of OMP2 proteins can be generated by immunizing rabbits, goats, or other suitable animals according to standard protocols such as those described in “Antibodies, A Laboratory Manual” (E. Harlow and D. Lane, Cold Spring Harbor Laboratory Press, 1988, 726 pp.) and “Using Antibodies, A Laboratory Manual” (E. Harlow and D. Lane, Cold Spring Harbor Laboratory Press, 1999, 495 pp.), which are herein incorporated by reference in their entirety. Antisera from the immunized animals are tested periodically for reactivity against antigen (such as single or multiple OMP2 proteins) by any appropriate method, such as by enzyme-linked immunosorbent assay (ELISA) in a microtiter plate format. The dilution factor at which no antigen-antibody binding can be observed is termed the antibody titer. A higher titer (that is, a higher dilution factor) indicates a stronger immune response against the immunogen. When a sufficient titer is reached, the antibody is then isolated from the crude serum. Isolated or purified antibodies can be further characterized by ELISA assays, dot blots, Western blots, or any other appropriate test method. Suitable antibodies can be incorporated, as single antibodies or as mixtures, into diagnostic test devices designed to detect a target (for example, NTHi) in a sample.

To generate anti-OMP2 antibodies in rabbits, purified OMP2 protein from one NTHi strain (51997) and OMP2 proteins from a mixture of three NTHi strains (51997, 49401, 49766) were used as a single immunogen and as multiple immunogens, respectively, in separate rabbit immunization programs for the generation of polyclonal antibodies. Five rabbits were immunized for each program.

A typical immunization protocol is as follows. Rabbits were inoculated on day 1 at four subcutaneous sites with an initial inoculation of 100 micrograms immunogen in 200 microliters phosphate-buffered saline (PBS) mixed with 200 microliters of complete Freund's adjuvant. Rabbits were bled on day 14, then given a first booster inoculation of 25 micrograms immunogen in PBS and incomplete Freund's adjuvant. On day 28, and monthly thereafter, the animals were bled and given an additional boost (identical to the first boost). Serum from each bleed was tested for titer, and when titers reached a sufficient level, the bleed/boost cycle was shortened from once monthly to once every 2 weeks. In cases of insufficient response (low titers), the boost inoculum was increased from 25 micrograms to 50 micrograms.

A similar immunization program was established in mice for the production of monoclonal antibodies. The primary immunization included 50 micrograms of immunogen diluted 1:1 with Titermax for a final volume of 500 microliters, administered subcutaneously into two sites per mouse. Boosts were performed with 25 micrograms immunogen diluted 1:1 with Titermax for a final volume of 500 microliters, administered subcutaneously into two sites per mouse.

For the initial polyclonal immunoglobulin G (IgG) purification step, a 45% saturated ammonium salt (SAS) cut was performed on the polyclonal serum. The same initial antibody purification step can be used for monoclonal ascites or tissue culture supernatants. The crude antibody preparation was further affinity purified with the appropriate OMP2 protein crosslinked to Pierce Amino-Link Coupling Gel (catalogue number 20501, Pierce Biotechnology, Inc., Rockford, Ill.) according to the manufacturer's instructions for pH 10 coupling and blocking. OMP2 proteins were individually crosslinked according to strain to the affinity matrix, and the different strain-specific affinity matrices mixed to yield a mixed-strain OMP2 affinity column. An alternative method can include the use of the individual strain-specific affinity matrices used separately and, optionally, serially. These approaches were designed to yield affinity-purified polyclonal antibodies that were cross-reactive to all strains as well as affinity-purified polyclonal antibodies that were specific to a given OMP2 strain. Such antibodies would useful, for example, in phage display studies to determine sequences of OMP2 that are conserved between strains versus sequences that are strain-specific, or in mimotope design studies. Affinity purification of the crude anti-OMP2 antibodies was carried out using phosphate-buffered saline for the running buffer, acid glycine (pH 2.4) for the elution buffer, and 0.5 molar sodium phosphate as the neutralization buffer. The affinity-purified antibodies were dialyzed against PBS and concentrated using Amicon Stir-Cells with YM30 membranes or with Amicon Centricons or Centripreps with YM30 or YM10 membranes (Millipore, Inc., Billerica, Mass., USA)

Other suitable affinity purification supports can be prepared and used for antibody purification as is known in the art (Pierce Technical Handbook, Pierce Biotechnology, Inc., 1994, Rockford, Ill.; Pierce 2003-2004 Applications Handbook and Catalogue, Pierce Biotechnology, Inc., 2003, Rockford, Ill.).

### Example 3

### Antibody Characterization and Selection

This example describes a non-limiting embodiment of a method for characterizing and selecting polyclonal antibodies raised against single or multiple immunogens.

Polyclonal antibodies were prepared against a single immunogen (purified OMP2 protein from non-typeable Haemophilus influenzae (NTHi) strain 51997) and against multiple immunogens (OMP2 proteins from a mixture of NTHi strains 51997, 49401, and 49766) in rabbits as described in Example 2.

Test bleeds were taken from the immunized rabbits at 6 weeks post primary immunization for the OMP2 multiple immunogen program and on the same date for the OMP2 single immunogen (strain 51997) program. Rabbits had each received three boosts after the primary immunization. Test bleed titers were compared in an ELISA plate format, and the data from the top three responders from each group of five rabbits is given in Table 2.

A typical ELISA procedure is as follows. The experiments were carried out using disposable, non-sterile, easy-wash, flat-bottom 96-well plates (catalogue number 25883-96, Corning, Inc., NY, USA). For testing using whole cells as antigen, each well was coated with each well was coated with 100 microliters of a suspension of 1×10⁸ non-typeable Haemophilus influenzae (NTHi) whole cells per milliliter phosphate-buffered saline (PBS). Several strains were used, with each strain placed on a separate ELISA plate. Plates were incubated 2 hours at room temperature, or alternatively, overnight at 4 degrees Celsius. The remaining solution in the wells was discarded, and the wells washed twice with PBS containing 0.05% Tween-20 (PBST) and blocked with 1% bovine serum albumin in PBS for at least 1 hour at room temperature. The blocking solution was discarded, 100 microliters of antiserum (diluted as desired) was added to each well and the plate incubated 1 hour at room temperature. The plate was washed 4 times with PBST, and 100 microliters of appropriately diluted secondary antibody (anti-rabbit IgG conjugated to horseradish peroxidase) added to each well. The plate was incubated 1 hour at room temperature, washed 4 times with PBST, and incubated with 100 microliters of the peroxidase substrate 3,3′,5,5′-tetramethylbenzidine (TMB) until sufficient signal is obtained (generally 10 to 15 minutes). The signal was read with a microplate reader at 630 nanometers. Pre-immunization test bleed sera were used as negative controls.

The dilution factor at which no anti-OMP2 antibody binding could be detected was termed the antibody titer. Table 2 shows results of three sets of titrations (taken from different test bleeds) from the top three responders of each immunization program. The data indicated that, in comparison to the 51997 OMP single immunogen program, the 51997/49401/49766 OMP2 multiple immunogen program generated antisera with higher overall cross-reactivity and higher overall titers against the seven OMP2 capture antigens tested. This enhanced cross-reactivity and titer was observed as early as six weeks into the immunization program, and improved with time as the immunization program progressed.

TABLE 2
ELISA titrations of test bleeds from rabbits immunized with single
or multiple immunogen preparations derived from non-typeable Haemophilus influenzae OMP2
proteins. Titer values given in/1000.

| | | | Capture Antigen | | | | | | |
|---|---|---|---|---|---|---|---|---|---|
| Bleed | Immunization | Rabbit | OMP2 | ATCC Strain | | | | | |
| Date | Program | ID | 51997 | 49401 | 49766 | 43163 | 19418 | 33540 | 53600 |
| 7 Jul. | Single | A1033 | 128 | 128 | 512 | 128 | 128 | 512 | 32 |
| 2003 | OMP2 | A1037 | 128 | 128 | 512 | 64 | 64 | 512 | 32 |
| | (51997) | A1038 | 128 | 128 | 512 | 128 | 642 | 256 | 32 |
| | Multiple | B1239 | 128 | 256 | 512 | 128 | 128 | 512 | 128 |
| | OMP2 | B1246 | 512 | 512 | >512 | 64 | >512 | >512 | 64 |
| | (51997/49401/ | B1247 | 128 | 512 | >512 | 512 | >512 | >512 | 256 |
| | 49766) | | | | | | | | |
| October | Single | A1033 | 160 | 160 | 80 | 160 | nd* | 160 | 20 |
| 2003 | OMP2 | A1034 | 80 | 80 | 40 | 80 | 20 | 80 | 10 |
| | (51997) | A1036 | 80 | 160 | 40 | 160 | 40 | 160 | 20 |
| | Multiple | B1239 | 160 | 320 | 160 | 320 | 80 | 320 | 80 |
| | OMP2 | B1246 | 640 | 640 | 640 | 1280 | 160 | 1280 | 160 |
| | (51997/49401/ | B1247 | 640 | 640 | 1280 | 640 | nd* | 640 | 80 |
| | 49766) | | | | | | | | |
| November | Single | A1033 | 160 | 80 | 40 | 160 | 40 | 160 | 20 |
| 2003 | OMP2 | A1034 | 80 | 80 | 80 | 80 | 20 | 160 | 10 |
| | (51997) | A1036 | 80 | 160 | 40 | 80 | 20 | 160 | 20 |
| | Multiple | B1239 | 160 | 160 | 160 | 320 | 40 | 320 | 80 |
| | OMP2 | B1246 | 320 | 640 | 320 | 640 | 80 | 1280 | 80 |
| | (51997/49401/ | B1247 | 640 | 640 | 1280 | 640 | 160 | 640 | 80 |
| | 49766) | | | | | | | | |

*nd, not determined

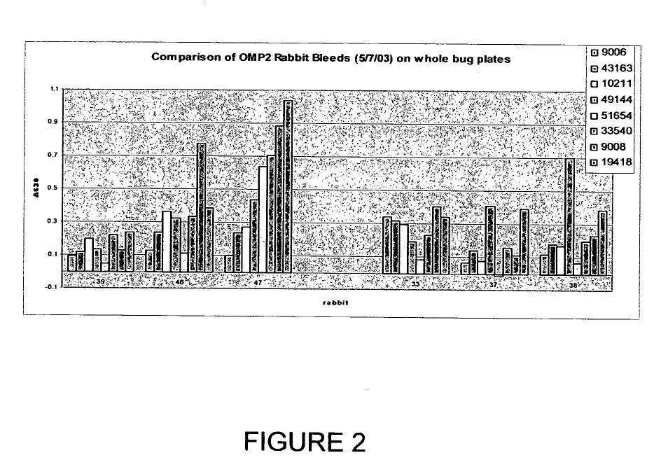

Additional assays on the test bleeds from 5 Jul. 2003 were performed using ELISA capture plates coated with intact bacterial cells of eight Haemophilus influenzae strains, including the typeable H. influenzae strains 9006 (type a), 9008 (type d), 10211 (type b), and 51654 (type b) (FIG. 2). The response of the three strongest responders of each program was compared in a titration assay against the seven OMP2 capture antigens. Higher overall titers and thus higher overall cross-reactivity were again observed for antibodies generated from the 51997/49401/49766 OMP2 multiple immunogen program than was observed for antisera from the 51997 OMP2 single immunogen program.

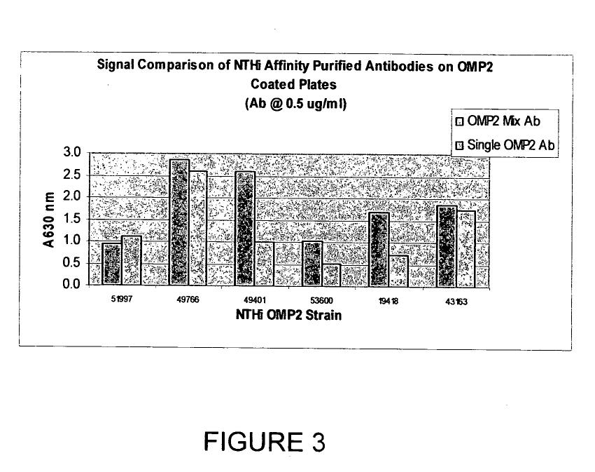

A 45% saturated ammonium salt cut was performed on the same set of test bleeds (5 Jul. 2003) from each immunization program. The resulting salt cut was further purified on either a single-strain OMP2 or multi-strain OMP2 affinity column; the latter consisted of a combination of affinity matrices individually prepared for each OMP2 strain used in the multiple immunogen program. The purified antibodies were characterized by ELISA titration. Briefly, ultra-high binding polystyrene microtiter strip assemblies (Immulon-4 HBX, part number 6405, Thermo Labsystems, Franklin, Mass., USA) were coated with purified OMP2 proteins from six NTHi strains (51997, 49766, 49401, 53600, 19418, and 43163) at 20 nanograms per well using standard protocols (similar to those described in the preceding ELISA protocol). Affinity-purified antibodies were then allowed to bind to the immobilized antigens. Bound primary antibody (anti-OMP2) was detected with an enzyme-labelled secondary antibody (goat anti-rabbit IgG conjugated to horseradish peroxidase (catalogue number 170-6515, BioRad Laboratories, Hercules, Calif., USA). Plates were developed with the peroxidase substrate 3,3′,5,5′-tetramethylbenzidine (TMB, prepared in-house or purchased from Moss, Inc, Pasadena, Md.) and absorbance measured at 630 nanometers. A representative graph of signal at one antibody concentration is presented in FIG. 3. Using equivalent molar amounts of affinity-purified anti-OMPs antibody, greater overall signal was generally observed for antibodies generated from the 51997/49401/49766 OMP2 multiple immunogen program than from the 51997 OPM2 single immunogen program.

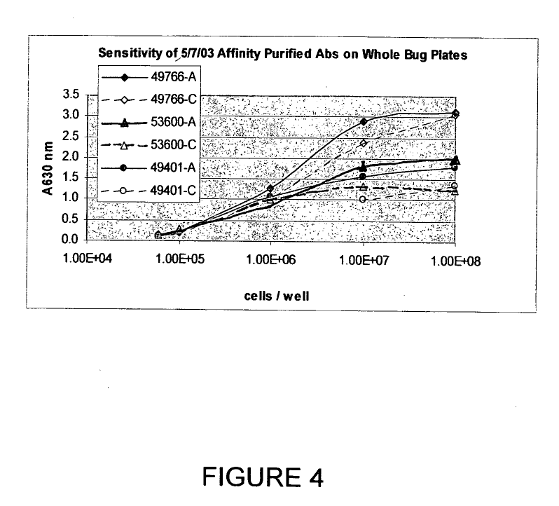

The affinity-purified antibodies from the same set of test bleeds (5 Jul. 2003) were also tested on microtiter plates coated with whole non-typeable Haemophilus influenzae cells (strains 49766, 53600, and 49401) (FIG. 4). At cell concentrations of about 10⁷ to about 10⁸ cells per well, the observed signals were stronger for antibodies generated from the 51997/49401/49766 OMP2 multiple immunogen program than for antibodies generated from the 51997 OPM2 single immunogen program. At cell concentrations of about 10⁴ to about 10⁶ cells per well, antibody was in excess and the observed signals were about equivalent.

Several subsequent lots of antibody were prepared and affinity-purified as described above from later bleeds in both immunization programs. These selected antibodies are usable for any desirable purpose, including immunochemical diagnosis of infectious disease states in human subjects. In a non-limiting example of a kit and assay method employing the selected antibodies of improved cross-reactivity, the affinity-purified antibodies were incorporated into a proprietary immunochromatographic membrane assay (ICT, Binax, Inc., Portland, Me.) (see U.S. Pat. No. 5,877,028, “Immunochromatographic assay device”, to Chandler et al., issued 2 Mar. 1999, which is incorporated by reference in its entirety herein). Suitable combinations of antibodies at appropriate concentrations can be determined for a given application and assay or device format by experimental testing, for example, by testing different antibody formulations on samples of known strains of non-typeable Haemophilus influenzae to obtain a desirable level of assay sensitivity and specificity.

The proprietary immunochromatographic assay device includes a nitrocellulose membrane onto which the test antibodies were permanently immobilized by adsorption as a narrow stripe (“sample line” or “test line”). A quantity of the same test antibodies were conjugated to visualizing gold particles and temporarily immobilized by drying onto an inert fibrous support (“conjugate pad”). The conjugate pad and the striped nitrocellulose membrane are combined into a test strip mounted on one side of a hinged, book-shaped device, which also contains a sample application site (containing an extraction pad) on the side opposite to the test strip. A sample was added to the extraction pad and the book-shaped device was closed. The gold-conjugated antibodies in the conjugate pad specifically bound antigen (NTHi OMP2) present in the sample, forming antigen-antibody complexes. The antigen-antibody complexes travelled further along the test strip to be captured by the antibody immobilized in the sample line of the test strip, forming a visually detected signal (a pink-to-purple colored line) when sufficient complex is formed. Test results were positive if a pink-to-purple colored line appeared on the sample line, and negative if no pink-to-purple colored line appeared on the sample line. In another embodiment of a suitable immunochromatographic test device, an in-line format is used, wherein the sample is applied to an extraction pad that is on one end of the test strip. The sample fluid travels along the strip to the conjugate pad and then the striped nitrocellulose membrane, with the fluid flow aided by wicking by an absorbent pad on the end of the nitrocellulose membrane opposite to that where the conjugate pad is located.

Antibodies generated from the 51997/49401/49766 OMP2 multiple immunogen program and antibodies generated from the 51997 OPM2 single immunogen program were tested for sensitivity against several NTHi strains using the immunochromatographic membrane assay (Table 3). Antibodies generated from the 51997/49401/49766 OMP2 multiple immunogen program possessed improved cross-reactivity and sensitivity for multiple strains of NTHi, relative to antibodies generated from the 51997 OPM2 single immunogen program.

TABLE 3
Comparison of Rabbit Anti-NTHi Antibodies
in Binax ICT Test Devices.

| sample (NTHi): | | Mix Strain OMP2 Ab: | | | Single Strain OMP2 | |
|---|---|---|---|---|---|---|
| | conc | | | Ab # | Ab: | |
| strain | (cells/mL) | Ab#1 | Ab#2 | 1&2 | Ab#1 | Ab#2 |
| 51997*^{,}** | 1 × 10⁸ | ++++ | ++++ | ++++ | +++ | +++ |
| | 1 × 10⁷ | +++ | + | ++ | + | +++ |
| | 1 × 10⁶ | wk+ | − | − | wk+ | ++ |
| 49401** | 1 × 10⁸ | ++++ | ++++ | ++++ | − | − |
| | 1 × 10⁷ | +++ | + | ++ | − | − |
| | 1 × 10⁶ | + | − | − | − | − |
| 53600 | 1 × 10⁸ | ++++ | ++ | +++ | − | − |
| | 1 × 10⁷ | ++ | + | + | − | − |
| | 1 × 10⁶ | + | − | − | − | − |
| 53775 | 1 × 10⁸ | nd | +++ | +++ | nd | + |
| | 1 × 10⁷ | nd | − | wk+ | nd | − |
| | 1 × 10⁶ | nd | − | − | nd | − |
| 49766** | 1 × 10⁸ | ++++ | nd | nd | + | nd |
| | 1 × 10⁷ | ++ | nd | nd | − | nd |
| | 1 × 10⁶ | − | nd | nd | − | nd |

*strain used for both single and multiple OMP2 immunogen preparation
**strains used in multiple OMP2 immunogen preparation
Intensity of positive response is indicated by the number of pluses (+), where ++++ indicates a very strong positive, and wk+ indicates the lowest visually detectable positive; negative response is designated by a minus symbol (−); nd, not determined.

### Example 4

### Phage Display for Identification of Conserved Antigenic Sites

This example describes a non-limiting embodiment of a method for improving the affinity of selected antibodies raised against single or multiple immunogens. In this example, phage display is used to identify conserved antigenic sites in non-typeable Haemophilus influenzae (NTHi) OMP2 proteins.

Preparation of Monoclonal Antibodies: Mice are immunized with OMP2 antigen in a suitable form (for example, with crude or purified OMP2 proteins or with intact or lysed NTHi cells) from NTHi strains of interest, using immunization protocols known in the art (see, for example, the protocol described in Example 2, or “Monoclonal Antibodies: A Practical Approach”, P. Shepherd and C. Dean, editors, Oxford University Press, 2000, 479 pp., which is incorporated by reference in its entirety herein). When significant antibody titer is obtained (as evidenced by high titers of test bleeds from immunized animals), isolated spleen cells from the immunized mice are fused to a melanoma tumor cell line using standard protocols (Kohler and Milstein (1975), Nature, 256:495-497). In screening clones, clones that are crossreactive on most or all of the NTHi strains of interest (as determined by techniques such as ELISA, dot blot, Western blots etc.) are selected.

Selected clones are then grown in cell culture or as ascites. Monoclonal antibodies are then affinity purified from the cell culture supernatant or ascites. Salt cuts (45-50%) may be performed for crude isolation of antibodies. Further purification is then done with affinity columns, as described above under Example 2. Protein A, protein G, OMP2 affinity matrices, or a combination thereof, may used for affinity column(s).

Preparation of Polyclonal Antibodies: Polyclonal antibodies are generated as described above under Example 2 or using other suitable techniques known in the art (“Antibodies: A Laboratory Manual”, E. Harlow and D. Lane, editors, Cold Spring Harbor Laboratory, 1988, 726 pp). Antibodies that are cross reactive are selected and purified by serially passing antisera or salt precipitated crude immunoglobulin over multiple OMP2 affinity columns (each specific to one NTHi strain). For example, crude immunoglobulin from an ammonium sulphate precipitation can be passed over an NTHi strain 51997 OMP2 affinity column, then the eluted antibody passed over an NTHi strain 49107 OMP2 affinity column, then the eluted antibody passed over a third NTHi strain OMP2 affinity column. By using multiple passages over different strain OMP2 columns, polyclonal antibodies that are cross reactive to many or most OPM2 strains are obtained.

Optionally, a negative selection step to help eliminate antibodies crossreactive to undesirable antigens (such as other bacteria) may be performed. For example, antisera can be passed over an affinity column made from membrane proteins or other antigen from a bacterium other than non-typeable Haemophilus influenzae (such as Moraxella catarrhalis outer membrane proteins). The unretained antibodies are then purified over NTHi OMP2 columns.

Immobilization of Antibodies: Selected antibodies may be used free in solution, or may be temporarily or permanently immobilized, directly or indirectly, onto a separate moiety, molecule, molecular structure, matrix, or surface. For panning, antibodies are generally immobilized onto a solid phase, for example, by covalent or non-covalent coupling or by passive adsorption. Purified antibodies may be buffer-exchanged or dialyzed into an appropriate coating buffer (for example, phosphate buffered saline or sodium carbonate buffer) for covalent or non-covalent adsorption onto a solid phase such as beads or plates. Various solid phases can be utilized for panning, such as polystyrene microtiter plates (Costar, Cambridge, Mass.), magnetic particles (Promega), carboxylate-modified latex particles (Polysciences, Inc., Warrington, Pa.) or amide-modified latex particles (Bangs Laboratories, Inc., Fishers, Ind.). Antibody may be initially coupled to another molecule, such as biotin and then bound to Avidin, NeutrAvidin or StrepAvidin (Pierce) coated solid support. Antibodies may be covalently or non-covalently immobilized onto the solid phase.

In one non-limiting example, antibodies are coated onto latex particles by passive adsorption. Affinity-purified antibody solutions are incubated with the latex particles using end-over-end mixing overnight at 4 degrees Celsius. Particles are washed with phosphate-buffered saline containing 0.05% Tween-20 (PBST), by centrifuging the particle suspension at 14,000 times gravity in a microfuge for 5 minutes, discarding the supernatant, resuspending the particle pellet in PBST, and centrifuging again. This wash cycle is repeated twice more for a total of three washes. Following the final wash, particles are suspended in phosphate-buffered saline containing 2% bovine serum albumin as a blocking solution for 2 hours at room temperature with end-over-end mixing. Particles are washed three times with PBST, then resuspended in Tris-buffered saline containing 0.1% Tween-20 (TBST) for use in phage panning.

Phage Panning: Any suitable peptide or protein library, including libraries of linear or cyclic peptides or proteins, made in-house or purchased from commercial vendors, may be used for phage panning. In one non-limiting example, a Ph.D-12 Phage display Peptide Library Kit (catalogue number E8110S, New England BioLabs, Beverly, Mass.) is used and the manufacturer's protocol followed. The affinity-purified antibody-coated latex particle suspension, diluted in TBST (1.0 milliliter), is transferred to a 1.5-milliliter microcentrifuge tube. Phage (4×10¹⁰) is added. Phage may optionally be first negatively selected by pre-incubation with particles coated with another antibody, such as the flow-through antibodies from the polyclonal affinity purification step against non-typeable Haemophilus influenzae (NTHi) OMP2 proteins, or antibodies directed against another bacterial surface protein (for example, Moraxella catarrhalis outer membrane proteins). In this case, after 1 hour of preincubation, the particle suspension is centrifuged and the unbound phage in the supernatant transferred to the microcentrifuge tube containing the NTHi OMP2 antibody-coated latex particles for the first panning step. The suspension is mixed, end-over-end, 1 hour at room temperature.

The non-binding phage is discarded by microcentrifuging the suspension and discarding the supernatant. Phage bound to the NTHi OMP2 antibody-coated latex particles are washed a minimum of 4 to 5 times with TBST, and resuspended in TBST. Bound phage is eluted off the particles with acid glycine, pH 2.2 for 5 minutes. The glycine elution is centrifuged and the supernatant transferred to a fresh tube and neutralized with Tris-HCl (pH 9.1).

The eluted phage is amplified in E coli (ER2738) culture. The culture is incubated for 4.5 hours with vigorous shaking, followed by centrifugation. The supernatant is transferred to a fresh tube and re-centrifuged. The supernatant is transferred to a fresh tube and the phage precipitated by addition of one-sixth volume PEG/NaCl (20% polyethylene glycol—8000, 2.5 molar sodium chloride). The mixture is incubated overnight at 4 degrees Celsius, centrifuged 15 minutes at 10,000 rpm at 4 degrees Celsius, then resuspended in TBS. The phage is reprecipitated with one-sixth volume PEG/NaCl for 15 to 60 minutes on ice, then centrifuged. The supernatant is discarded and the pellet suspended in 200 microliters TBS containing 0.02% sodium azide. The amplified phage is titered following procedures suggested by the peptide library's manufacturer (New England BioLabs).

The panning and amplification steps are repeated 2 or 3 more times, with the Tween-20 concentration increased with each successive panning step, up to 0.5% volume/volume. The unamplified final panning eluate is titered. Ten to fifteen plaques are selected for characterization of binding. Each clone is transferred to a separate diluted culture tube, and incubated at 37 degrees Celsius for 4.5 to 5 hours. The cultures are centrifuged, and the amplified supernatant analyzed by ELISA following procedures suggested by the peptide library's manufacturer (New England BioLabs).

Positive-binding clones are identified by utilizing horseradish peroxidase-conjugated anti-M13 antibody (catalogue number 27-9411-01, Pharmacia). Positive clones are amplified. DNA purified from the amplified clones is sequenced and the sequences compared in order to determine consensus sequences. Peptides to serve as putative mimotopes or epitopes can be synthesized based on the determined consensus sequences. Such peptides are of use, for example, as immunogens for the production of antibodies, for affinity purification of antibodies, for vaccines or other therapeutics, or for use in diagnostic kits.

All headings are for the convenience of the reader and should not be used to limit the meaning of the text that follows the heading, unless so specified. Various changes and departures may be made to the present invention without departing from the spirit and scope thereof. Accordingly, it is not intended that the invention be limited to that specifically described in the specification or as illustrated in the drawings, but only as set forth in the claims.

1. A method for generating at least one antibody having improved cross-reactivity for an infectious organism that exists in more than one immunotype, wherein said immunotypes are due to at least one antigenic variation, said method comprising the steps of:
a) providing multiple immunogen preparations derived from said at least one antigenic variation;
b) immunizing at least one animal with said multiple immunogen preparations;
c) selecting at least one antibody from said immunized at least one animal;
wherein said selected at least one antibody is of improved cross-reactivity for said infectious organism, relative to antibodies from animals immunized with a single immunogen derived from said at least one infectious organism.

2. The method of claim 1, wherein said selected at least one antibody is polyclonal.

3. The method of claim 1, wherein said selected at least one antibody is monoclonal.

4. The method of claim 1, wherein said selected at least one antibody is an antibody fragment.

5. The method of claim 1, wherein said at least one animal is a mammal.

6. The method of claim 1, wherein said at least one animal is a bird.

7. The method of claim 5, wherein said mammal is a rabbit.

8. The method of claim 5, wherein said mammal is a mouse.

9. The method of claim 5, wherein said mammal is a goat.

10. The method of claim 5, wherein said mammal is a sheep.

11. The method of claim 5, wherein said mammal is a horse.

12. The method of claim 5, wherein said mammal is a non-human primate.

13. The method of claim 5, wherein said mammal is a human.

14. The method of claim 1, wherein said infectious organism is non-typeable Haemophilus influenzae.

15. The method of claim 14, wherein said multiple immunogen preparations are derived from OMP2 protein from multiple non-typeable Haemophilus influenzae OMP2 immunotypes.

16. The method of claim 15, wherein said OMP2 protein comprises OMP2 protein isolated from cell outer membrane.

17. The method of claim 15, wherein said OMP2 protein comprises OMP2 protein not isolated from cell outer membrane.

18. The method of claim 15, wherein said OMP2 protein comprises at least one OMP2 protein fragment.

19. The method of claim 15, wherein said OMP2 protein comprises a recombinant protein.

20. The method of claim 15, wherein said OMP2 protein comprises a fusion protein.

21. The method of claim 15, wherein said OMP2 protein comprises a mimotope.

22. The method of claim 14, wherein said multiple non-typeable Haemophilus influenzae OMP2 strains comprise between 2 to 4 strains.

23. The method of claim 14, wherein said multiple non-typeable Haemophilus influenzae OMP2 strains comprise 5 or more strains.

24. The method of claim 1, further comprising improving the affinity of said selected at least one antibody for said infectious organism.

25. The method of claim 24, wherein said improving comprises use of affinity separation.

26. The method of claim 24, wherein said improving comprises use of display technology.

27. A method to detect an infectious organism that exists in more than one immunotype, wherein said immunotypes are due to at least one antigenic variation, said method comprising the steps of:
a) providing a sample suspected of containing said at least one antigenic variation;
b) providing at least one antibody generated by the method of claim 1;
c) contacting said sample with said at least one antibody, under conditions that allow said at least one antibody to bind to and form a complex with said at least one antigenic variation;
d) detecting said complex, wherein said detection is positive if concentration of said infectious organism in said sample is greater than or equal to than a reference concentration, and said detection is negative if concentration of said infectious organism in said sample is less than said reference concentration.

28. The method of claim 27, wherein said selected at least one antibody is polyclonal.

29. The method of claim 27, wherein said selected at least one antibody is monoclonal.

30. The method of claim 27, wherein said selected at least one antibody is an antibody fragment.

31. The method of claim 27, wherein said at least one animal is a mammal.

32. The method of claim 27, wherein said at least one animal is a bird.

33. The method of claim 31, wherein said mammal is a rabbit.

34. The method of claim 31, wherein said mammal is a mouse.

35. The method of claim 31, wherein said mammal is a goat.

36. The method of claim 31, wherein said mammal is a sheep.

37. The method of claim 31, wherein said mammal is a horse.

38. The method of claim 31, wherein said mammal is a non-human primate.

39. The method of claim 31, wherein said mammal is a human.

40. The method of claim 27, wherein said infectious organism is non-typeable Haemophilus influenzae.

41. The method of claim 40, wherein said multiple immunogen preparations are derived from OMP2 protein from multiple non-typeable Haemophilus influenzae OMP2 immunotypes.

42. The method of claim 41, wherein said OMP2 protein comprises OMP2 protein isolated from cell outer membrane.

43. The method of claim 41, wherein said OMP2 protein comprises OMP2 protein not isolated from cell outer membrane.

44. The method of claim 41, wherein said OMP2 protein comprises at least one OMP2 protein fragment.

45. The method of claim 41, wherein said OMP2 protein comprises a recombinant protein.

46. The method of claim 41, wherein said OMP2 protein comprises a fusion protein.

47. The method of claim 41, wherein said OMP2 protein comprises a mimotope.

48. The method of claim 40, wherein said multiple non-typeable Haemophilus influenzae OMP2 strains comprise between 2 to 4 strains.

49. The method of claim 40, wherein said multiple non-typeable Haemophilus influenzae OMP2 strains comprise 5 or more strains.

50. The method of claim 27, further comprising improving the affinity of said selected at least one antibody for said infectious organism.

51. The method of claim 50, wherein said improving comprises use of affinity separation.

52. The method of claim 50, wherein said improving comprises use of display technology.

53. The method of claim 27, wherein said at least one antibody is used in more than one form.

54. The method of claim 27, wherein said at least one antibody comprises a detectable label.

55. The method of claim 27, wherein said at least one antibody comprises a functional group.

56. The method of claim 27, wherein said at least one antibody is further capable of binding to at least one mimotope that mimics said at least one antigenic variation derived from said infectious organism.

57. The method of claim 27, wherein said positive detection is optionally at least semi-quantitative.

58. The method of claim 27, wherein said method further comprises simultaneous or parallel detection of more than one infectious organism.

59. The method of claim 27, wherein said at least one antigenic variation is modified.

60. A kit for performing the method of claim 27.

61. A method for generating a selection of antibodies having improved cross-reactivity for an infectious organism that exists in more than one immunotype, wherein said immunotypes are due to at least one antigenic variation, said method comprising the steps of:
a) providing multiple immunogen preparations derived from said at least one antigenic variation;
b) immunizing multiple groups of animals, wherein each of said groups comprises at least one animal, and wherein each of said animals is immunized with a single immunogen preparation;
c) selecting at least one antibody from each of said groups; and
d) combining said selected antibodies,
wherein said combination of selected antibodies is of improved cross-reactivity for said infectious organism.
